(19) United States
(12) Patent Application Publication
(10) Pub. No.: US 2013/0230857 A1
(43) Pub. Date: Sep. 5, 2013

(54) HYBRID SELECTION USING GENOME-WIDE BAITS FOR SELECTIVE GENOME ENRICHMENT IN MIXED SAMPLES

(71) Applicants: Andreas Gnirke, Wellesley, MA (US); Peter Rogov, Winchester, MA (US); Daniel Neafsey, Newton, MA (US); Chad Nusbaum, Newton, MA (US); Alexandre Melnikov, Bellingham, MA (US)

(72) Inventors: Andreas Gnirke, Wellesley, MA (US); Peter Rogov, Winchester, MA (US); Daniel Neafsey, Newton, MA (US); Chad Nusbaum, Newton, MA (US); Alexandre Melnikov, Bellingham, MA (US)

(73) Assignee: The Broad Institute, Inc., Cambridge, MA (US)

(21) Appl. No.: 13/883,422

(22) PCT Filed: Nov. 3, 2011
(86) PCT No.: PCT/US11/59149

§ 371 Date: May 3, 2013

Related U.S. Application Data
(60) Provisional application No. 61/410,713, filed on Nov. 5, 2010.
(60) Provisional application No. 61/484,019, filed on May 9, 2011.

(51) Int. Cl. C12Q 1/68

(52) U.S. Cl. CPC C12Q 1/6888
USPC 435/6.11; 536/24.32

(57) ABSTRACT

The present invention provides methods for sequencing and genotyping of DNA useful for analysis of samples in which the target DNA represents a small portion (e.g., 10-1000-fold less) that a contaminating DNA source. Accordingly, the methods described herein are useful for sequencing or genotyping pathogen DNA, such as malaria DNA, in clinical samples taken from infected subjects.

### STATEMENT AS TO FEDERALLY FUNDED RESEARCH

This invention was made with United States Government support under grant HHSN27220090018C awarded by the National Institute of Allergy and Infectious Diseases. The Government has certain rights to this invention.

### BACKGROUND OF THE INVENTION

The invention relates to methods for enriching genomes in samples that include contaminating DNA and methods for analyzing genomic DNA from such samples.

The falling cost of DNA sequencing means that sample quality, rather than expense, is now the blocking issue for many infectious disease genome sequencing projects. Pathogen genomes are generally very small relative to that of their human host, and are typically haploid in nature. Therefore, even a modest number of nucleated human cells present in infectious disease samples may result in the pathogen DNA representation being dwarfed relative to the host human DNA. This difference in representation poses a significant challenge to achieving adequate sequence coverage of the pathogen genome in a cost-effective manner. Separation of host and pathogen cells prior to DNA extraction can be difficult or inconvenient, particularly in field settings common to clinical trials in developing countries. The increasing use of genome-wide association studies to determine the genetic basis of important infectious disease phenotypes, such as drug resistance (Mu et al., Nat. Genet. 2010, 42:268-271), requires sequencing or genotyping hundreds to thousands of pathogen isolates, making a shortage of quality specimens an acute problem.

Existing methods for dealing with human DNA contamination in infectious disease samples typically require significant time, money, or special handling of samples at the time of collection.

Thus, there exists a need for improved methods for sequencing pathogen DNA in samples that contain host or other contaminating DNA.

### SUMMARY OF THE INVENTION

To address the problem of sequencing DNA in heterogeneous DNA samples, a solution hybrid selection approach useful for analysis of genomic DNA in samples that contain mixtures of genomic DNA from two or more species, (e.g., a biological sample taken from a subject infected with a pathogen, parasite or symbiont, or commensal organism) has been developed and is described below.

These approaches, in general, have been carried out using detectably labeled probes that provide coverage of the target organism genome. The baits are hybridized to the target organism genome in the heterogeneous sample and are separated from the contaminating DNA using a binding partner of the detectable label. The enriched DNA from the target organism is then sequenced. As exemplified below, two approaches to bait design have been used. The first approach involves generation of synthetic oligonucleotides that hybridize to specific regions of target organism genome, but do not target the contaminating DNA. The second approach involves the use of fragmented genomic DNA from the target organism as the bait sequence. In either approach, detectably labeled RNA generated from the DNA can be used as bait.

In one example, biotinylated RNA probes complementary to the pathogen genome are hybridized to pathogen DNA in solution and retrieved with magnetic streptavidin-coated beads. Host DNA is washed away, and the captured pathogen DNA is then eluted and amplified for sequencing or genotyping. This general method has been applied using two different approaches to bait design: (1) synthetic 140 base pair oligonucleotides targeting specific regions of the P. falciparum 3D7 reference genome assembly and (2) “whole genome baits” (WGB) generated from pure P. falciparum DNA. Using either protocol, significant enrichment of P. falciparum DNA was achieved, allowing for whole genome sequencing on samples which otherwise would have been prohibitively expensive to sequence.

Accordingly, in a first aspect, the invention features a method for enriching the genome of a target organism in a DNA sample that includes both contaminating DNA (e.g., host DNA, for example, mammalian DNA such as human DNA) and DNA of the target organism. The method includes (a) contacting the sample with at least 1,000 (e.g., at least 2,000, 3,000, 4,000, 5,000, 7,500, 10,000, 20,000, 30,000, 50,000, or 100,000) different, detectably-labeled hybridization bait sequences specific for the target DNA, under conditions in which the bait sequences hybridize to the target organism DNA but do not substantially hybridize to the contaminating DNA; and (b) selectively isolating the hybridized target DNA based on the detectable label, thereby enriching for the genome of the target organism. The method may further include step (c) genotyping or sequencing the isolated target DNA of step (b). The isolated target DNA of step (b) may be amplified using polymerase chain reaction (PCR). The DNA sample, prior to step (a) contacting, may be subject to shearing and end-labeling (e.g., using end labels that are suitable for sequencing or PCR amplification of the DNA).

In certain embodiments, most of the DNA in the DNA sample is contaminating DNA (e.g., the ratio of contaminating DNA to target DNA is at least 2:1, 4:1, 10:1, 15:1, 20:1, 30:1, 40:1, 60:1, 80:1, 100:1, 125:1, 150:1, 200:1, 250:1, 300:1, 400:1, or 500:1).

The hybridization bait sequences may be prepared from the whole genome of the target organism, for example, where the bait sequences are prepared by a method that includes fragmenting genomic DNA of the target organism (e.g., where the fragmented bait sequences are end-labeled with oligonucleotide sequences suitable for PCR amplification or DNA sequencing or where the bait sequences are prepared by a method including attaching an RNA promoter sequence to the genomic DNA fragments and preparing the bait by transcribing (e.g., using biotinylated polynucleotides) the DNA fragments into RNA). The bait sequences may be prepared from specific regions of the target organism genome (e.g., are prepared synthetically). In certain embodiments, the bait sequences are labeled with biotin, a hapten, or an affinity tag or the bait sequences are generated using biotinylated primers, e.g., where the bait sequences are generated by nick-translation labeling of purified target organism DNA with biotinylated deoxynucleotides. In cases where the bait sequences are biotinylated, the target DNA can be captured using a streptavidin molecule attached to a solid phase. The bait sequences may include adapter oligonucleotides suitable for PCR amplification, sequencing, or RNA transcription. The bait sequences may include an RNA promoter or are RNA molecules prepared from DNA containing an RNA promoter (e.g., a T7 RNA promoter).

The bait sequences may be 60-500 bp in length (e.g., 100-300 bp in length). In certain embodiments, prior to performing step (a), whole genome amplification is performed on the DNA sample. In certain embodiments the hybridization is carried out under high stringency conditions (e.g., at about 65° C.).

The target organism may be a eukaryote, a prokaryote (e.g., a bacterium), an archeal organism, or a virus (e.g., a DNA virus or an RNA virus). The bacterium may be a Gram-negative bacterium a Gram-positive bacterium, a mycobacterium, or a mycoplasma (e.g., any of those described herein). In particular embodiments, the target organism is selected from the group consisting of Plasmodium vivax, Plasmodium falciparum, Plasmodium ovale, Plasmodium malariae, Chlamydia trachomatis, Trypanosoma cruzi, and Wolbachia.

In certain embodiments, the DNA sample is a biological sample (e.g., a cell sample, blood sample, or a sample containing blood components). The sample may be taken from a human infected with, or suspected of being infected with, a parasite or pathogen.

The invention also features a method of genotyping or sequencing the genome of a target organism. The method includes sequencing at least a portion of the genome in a sample containing DNA from a target organism prepared according to the above aspect of the invention.

In another aspect, the invention features a method for preparing whole genome bait. The method includes (a) transcribing RNA from fragmented genomic DNA of an organism, the DNA containing adapter sequences (e.g., sequences suitable for PCR amplification) that include an RNA polymerase start site (e.g., a T7 RNA polymerase start site); and (b) detectably labeling the RNA, thereby preparing whole genome bait. The detectable labeling step may be performed in conjunction with the transcribing step. The fragmented genomic DNA may be sheared DNA. The fragmented genomic DNA may average 100-1000, 100-500, 125-400, 150-300, or about 250 bases in length. The detectable label may be, for example, biotin, a hapten, or an affinity tag. The organism may be, for example, any described herein. The invention also features a composition including whole genome baits produced by this method.

In another aspect, the invention features a composition including RNA molecules that are detectably labeled, are 100-1000 bases in length, and together cover at least 50% (e.g., at least 75%, 85%, 90%, 95%, 98%, 99%, 99.5%, 99.9% or even 100%) of the genome of a target organism. The invention also features a kit including (a) the composition; and (b) a solid phase, where a binding partner of the detectable label is attached to the solid phase.

In another aspect, the invention features a hybridization composition including: (a) RNA molecules that are detectably labeled, are 100-1000 bases in length, and together cover at least 50% (e.g., at least 75%, 85%, 90%, 95%, 98%, 99%, 99.5%, 99.9% or even 100%) of the genome of a target organism that corresponds to the genome of a target organism; (b) a DNA sample that includes contaminating DNA and genomic DNA of the target organism; and (c) a solid phase to which a binding partner of the detectable label on the RNA present in the composition is attached.

In another aspect, the invention features a kit including (a) fragmented genomic DNA where at least a portion of the fragments further include adapter sequences, the adapter sequences include an RNA polymerase start site; (b) an RNA polymerase that initiates transcription at the start site; and (c) a solid phase, where a binding partner of a detectable label is attached to the solid phase. The kit may further include detectably-labeled nucleotide molecules suitable for use in RNA transcription.

The solid phase in any of the above kits may be a bead or chromatographic column. Such kits may further include a solution suitable for hybridization of the whole genome baits or RNA molecules to a DNA sample, or a concentrate thereof. The kits may further include a wash solution suitable for washing non-specifically bound DNA from the solid phase, or a concentrate thereof. Further, any of the kits discussed herein may further include an elution solution suitable for removing specifically bound DNA from a solid phase, or a concentrate thereof.

In another aspect, the invention features a system for enrichment of genomic DNA of a target organism in a sample that includes both DNA of the target organism and contaminating DNA. The system includes at least 1,000 (or for example at least 2,000, 3,000, 4,000, 5,000, 7,500, 10,000, 20,000, 30,000, 50,000, or even 100,000) bait sequences specific for the target organism that are detectably labeled; a sample containing DNA of the target organism and contaminating DNA; and a solid phase including a binding partner of the detectable label.

In another aspect, the invention features a system for sequencing or genotyping genomic DNA of a target organism in a sample that includes both DNA of the target organism and contaminating DNA. The system includes at least 1,000 (or for example at least 2,000, 3,000, 4,000, 5,000, 7,500, 10,000, 20,000, 30,000, 50,000, or even 100,000) bait sequences specific for the target organism that are detectably labeled; a sample containing DNA of the target organism and contaminating DNA; reagents for preparing the sample for sequencing; a solid phase including a binding partner of the detectable label; and a sequencing apparatus.

By “contaminating DNA” is meant any DNA in a sample originating from a source other than the target organism DNA that is being analyzed. Contaminating DNA may originate from target organism's host from which the sample is obtained.

By “DNA sample” is meant any composition that contains DNA of the desired target organism. The DNA sample may be a biological sample or a cellular sample. The DNA sample may contain or may be a blood component.

By “biological sample” is meant any sample of biological origin. In certain embodiments, biological samples are cellular samples.

By “blood component” is meant any component of whole blood, including host red blood cells, white blood cells (e.g., lymphocytes), and platelets. Blood components also include, without limitation, components of plasma, e.g., proteins, lipids, nucleic acids, and carbohydrates.

By “cellular sample” is meant a sample containing cells or components thereof. Such samples include, without limitation, tissue samples (e.g., samples taken by biopsy from any organ or tissue in the body) and naturally-occurring fluids (e.g., blood, lymph, cerebrospinal fluid, urine, cervical lavage, and water samples), portions of such fluids, and fluids into which cells have been introduced (e.g., culture media, and liquefied tissue samples). The term also includes a lysate. Any means for obtaining such a sample may be employed in the methods described herein; the means by which the sample is obtained is not critical.

By “target organism” is meant any organism. In certain embodiments, the target organism is a pathogen, parasite, commensal organism, or symbiont.

By “host” is meant any organism that harbors another organism, such as a pathogen, parasite, commensal organism, or symbiont. Hosts may be human or non-human animals or (e.g., mammals or plants).

By “high stringency conditions” are meant any conditions under which target DNA (e.g., from a pathogen, parasite, commensal organism, or symbiont) substantially hybridizes to bait sequences, but the bait sequences do not substantially hybridize to contaminating DNA (e.g., host DNA) in the same sample. Those skilled in the art will may determine appropriate conditions for any given sample type according to standard methodologies. In one specific example, hybridization is conducted at 65° C. for 66 h. This is followed by one wash at RT for 15 min. with 0.5 ml 1×SSC/0.1% SDS, followed by three 10-min. washes at 65° C. with 0.5 ml pre-warmed 0.1×SSC/0.1% SDS, with re-suspension of the beads containing the target DNA once at each washing step. The skilled artisan may also develop suitable conditions with similar selectivity, depending on the particular sample chosen according to standard methods.

The present invention provides a cost effective manner for sequencing or performing other analysis of genomic DNA present in samples that contain contaminating DNA, e.g., a sample taken from a subject infected with a pathogen.

Although sequencing has become considerably less expensive in recent years, it remains financially impracticable to sequence pathogen genomes from biological samples at scale due to the gross excess of host DNA typically present. The simplest way to compensate for host DNA contamination is to augment sequencing coverage depth. However, this strategy can be costly for all but the most lightly contaminated samples. In contrast, the current cost of purification by hybrid selection using WGB, for example, is approximately $250 (US), which is roughly equivalent to the current cost of generating 20-fold coverage of the 23 Mb P. falciparum genome from pure template using a fraction of an Illumina HiSeq lane. For augmented coverage to be an affordable strategy relative to hybrid selection for a target coverage level of 40× in a genome of this size, samples must contain at least 50% pathogen DNA. This titer of parasite DNA is rarely found in biological samples unless white cell purification is performed prior to DNA extraction. For a more typical biological sample containing only 1% P. falciparum DNA, hybrid selection resulting in 40-fold enrichment enables 40× coverage depth for a dramatically lower total current price (˜$1,000) than deeper sequencing of the unpurified sample (˜$40,000).

The modest cost and high performance of this hybrid selection purification protocol can facilitate sequencing of archival biological samples of malaria parasites and other pathogens that were previously considered unfit for sequencing by any methodology. Indeed, this can enable sequencing of important samples stored on filter papers or diagnostic slides predating the spread of drug resistance or associated with historic outbreaks. This purification protocol also broadens the accessibility of sequencing for biological samples of infectious organisms for which in vitro culture is possible but costly or inconvenient, such as Class IV “select agents” recognized by the CDC. This protocol is not limited to pathogens or parasites, and should be equally useful in sequencing commensal or symbiotic organisms closely associated with their host, such as intracellular Wolbachia bacteria. The reduction in sample quality and quantity requirements permitted by this method simplifies protocol design in large-scale clinical studies and can help realize the benefits of inexpensive, massively parallel sequencing technologies for studying infectious diseases in diverse contexts.

Other features and advantages of the invention will be apparent from the following Detailed Description, the drawings, and the claims.

### BRIEF DESCRIPTION OF THE DRAWINGS

FIG. 1 is a schematic diagram showing an example of a hybridization strategy employed in the methods described herein.

FIG. 2 is a schematic diagram showing generation of bait sequences from WGB and purification of target DNA (e.g., parasite DNA) from a mixed sample containing both target DNA and contaminating DNA (e.g., host DNA).

FIG. 3 is a schematic diagram showing enrichment of malaria DNA in mixed samples containing both human and malaria genomic DNA using WGB for hybrid selection, either with or without WGA.

FIG. 4 is a schematic diagram showing a comparison between (1) synthetic (Agilent) baits, (2) WGB, and (3) WGB used in conjunction with whole genome amplification (WGA).

FIGS. 5a-5c are graphs showing sequencing coverage plots from a randomly chosen region of P. falciparum chromosome 1. FIG. 5a shows unamplified (dark gray line) and WGA (black line) WGB compared to pure P. falciparum (lighter gray outline). FIG. 5b shows unamplified (dark gray line) and WGA (black line) synthetic baits read coverage compared to pure P. falciparum (lighter gray outline). Black bars (under the peaks) indicate bait locations. FIG. 5c shows local % GC (in 140 bp windows). Black bars (bottom of graph) indicate exons.

FIG. 6 is a schematic diagram showing sequencing results of hybrid selection.

FIGS. 7a and 7b are graphs showing genome-wide sequencing coverage and composition. FIG. 7a shows coverage thresholds for unamplified (dark gray) and WGA (black) WGB compared to pure P. falciparum (gray outline) and simulated coverage from a non-hybrid selected mock clinical sample (lighter gray line, left side of graph). FIG. 7b shows genome-wide coverage as a function of % GC. The vertical black line represents average exonic % GC. The histogram (bottom) represents the density distribution of genome composition (right vertical axis). Lines depict coverage (left vertical axis) of pure P. falciparum DNA (lighter gray, highest line), as well as unamplified (darker gray, lower line) and WGA (black, middle line) hybrid selected samples initially containing 1% P. falciparum DNA.

FIG. 8 is a graph showing a principal component analysis (PCA) plot based on SNP calls produced from hybrid-selected and non-hybrid-selected samples. The hybrid selected clinical sample from Senegal (black, upper right) clusters with 12 previously sequenced Senegal samples (light gray). The hybrid selected 3D7 samples (black, lower right) cluster with the non-hybrid selected 3D7 sample (dark gray). P. falciparum isolates from India (darkest gray, middle top) and Thailand (four dark gray dots, top) are also represented.

### DETAILED DESCRIPTION

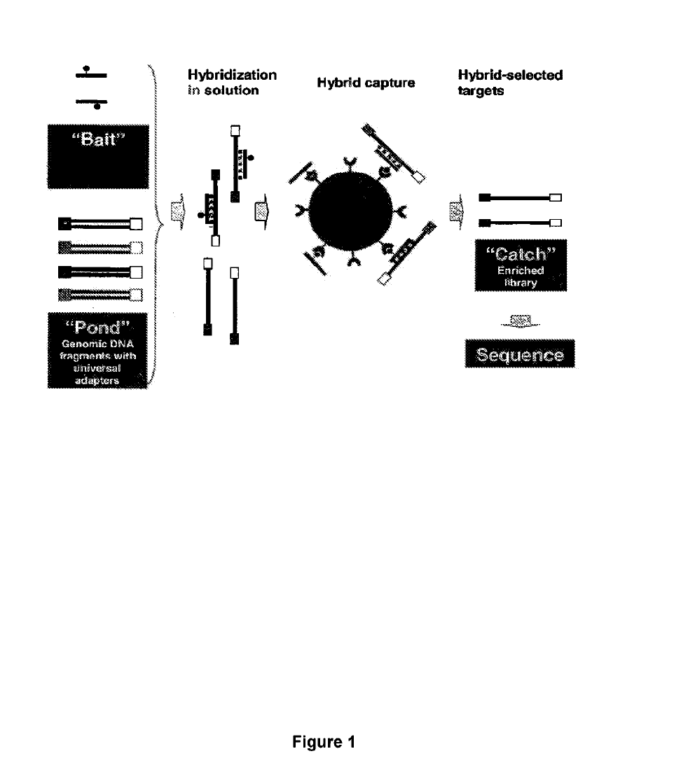

In general, the methods described herein involve generation of labeled bait sequences that cover all or a substantial portion of the target genome which are used to isolate and enrich the target DNA as compared to the contaminating or host DNA. This enriched sample is then suitable for sequencing using techniques known in the art. An exemplary strategy for hybridization is shown in FIG. 1.

As described below, hybrid selection was performed with two classes of bait (synthetic and WGB) on a mock clinical sample consisting of 99% human DNA and 1% Plasmodium DNA by mass, which falls within the range of DNA ratios found in many malaria clinical samples (Table 1). Hybridization and washing steps (described below) were carried out under standard high stringency conditions to reduce capture of contaminating, host DNA. The hybrid selection protocol requires a minimum of 2 μg of input DNA (combined host and pathogen), a quantity which may not be available from many types of field samples. Therefore, hybrid selection was also performed with both bait classes on 2 μg of WGA DNA generated from 10 ng of the mock clinical sample. Quantitative polymerase chain reaction (qPCR) analysis indicated that WGA does not significantly alter the fraction of malaria DNA present in the sample (post WGA % P. falciparum DNA=1.1+/−0.1).

TABLE 1
qPCR enrichment measurements from 12 clinical samples

| | | | Pre Hybrid | | |
|---|---|---|---|---|---|
| | | | Selection | Post Hybrid | |
| | % | | Parasite | Selection | |
| | Parasite | | [DNA] | Parasite [DNA] | Fold |
| Sample | DNA | WGA | (pg/μl) | (pg/μl) | Enrichment |
| Th231.08 | 0.11 | yes | 1.8 (0.6)^{a} | 71.1 (5.6) | 39.7 |
| (round 1) | | | | | |
| Th231.08 | 7.7 | no | 71.1 (5.6) | 349.1 (74.9) | 4.9 |
| (round 2) | | | | | |
| Th145.08 | 20 | no | 198.4 (17.4) | 477.6 (66.7) | 2.4 |
| Th032.09 | 12 | no | 114.7 (2.9) | 372.6 (59.3) | 3.2 |
| Th029.09 | 3 | no | 33.6 (0.8) | 317.3 (54.7) | 9.4 |
| Th093.09 | 2.8 | no | 28.5 (1.5) | 365.6 (53.4) | 12.8 |
| Th090.08 | 2.3 | no | 37.7 (1.1) | 300.4 (46.9) | 8.0 |
| Th139.08 | 2.1 | no | 23.6 (0.6) | 346.2 (50.7) | 14.7 |
| Th197.08 | 1.1 | no | 14.6 (0.0) | 222.7 (36.1) | 15.3 |
| Th140.08 | 0.99 | no | 9.6 (0.1) | 251.5 (37.4) | 26.2 |
| Th190.08 | 0.64 | no | 5.1 (0.2) | 218.7 (34.0) | 43.2 |
| Th238.08 | 0.53 | no | 6.7 (0.2) | 273.4 (38.1) | 41.0 |
| Th127.09 | 1.6 | no | 26.8 (0.4) | 368.5 (57.1) | 13.7 |
| Th175.08 | 48 | yes | 275.8 (7.2) | 556.9 (79.4) | 2.0 |

^{a}numbers in parentheses represent standard deviations

In summary, both bait strategies performed effectively and offer methods to sequence either targeted regions or complete genomes of pathogens in biological samples dominated by host DNA. Pairing this hybrid selection protocol with WGA further expands the range of biological samples now eligible for efficient pathogen genome sequencing. For example, for Plasmodium it is now possible to sequence the genome from dried blood spots on filter paper, an easily collectable and storable sample format.

### Target Organisms

The methods described herein employ any desired target organism. Exemplary target organisms include eukaryotic, a prokaryotic, and archeal organisms, and viruses (e.g., a DNA virus, or an RNA virus). Other exemplary target organisms that can be useful in the methods described herein are bacteria (e.g., Gram-negative bacteria or Gram-positive bacteria), mycobateria, mycoplasma, fungi, and parasitic cells. The organism may be a pathogen, a parasite, a commensal organism, or a symbiont.

Organisms difficult to culture ex vivo may be used in the methods described herein. Examples of such organisms include Plasmodium vivax, Chlamydia trachomatis, Trypanosoma cruzi, and Wolbachia. Other organisms that can be used in the described methods include Plasmodium falciparum, Plasmodium ovale, and Plasmodium malariae.

Examples of Gram-negative bacteria include, but are not limited to, bacteria of the genera, Salmonella, Escherichia, Chlamydia, Klebsiella, Haemophilus, Pseudomonas, Proteus, Neisseria, Vibro, Helicobacter, Brucella, Bordetella, Legionella, Campylobacter, Francisella, Pasteurella, Yersinia, Bartonella, Bacteroides, Streptobacillus, Spirillum, Moraxella, and Shigella. Particular Gram-negative bacteria of interest include, but are not limited to, Escherichia coli, Chlamydia trachomatis, Chlamydia caviae, Chlamydia pneumoniae, Chlamydia muridarum, Chlamydia psittaci, Chlamydia pecorum, Pseudomonas aeruginosa, Neisseria meningitides, Neisseria gonorrhoeae, Salmonella typhimurium, Salmonella entertidis, Klebsiella pneumoniae, Haemophilus influenzae, Haemophilus ducreyi, Proteus mirabilis, Vibro cholera, Helicobacter pylori, Brucella abortis, Brucella melitensis, Brucella suis, Bordetella pertussis, Bordetella parapertussis, Legionella pneumophila, Campylobacter fetus, Campylobacter jejuni, Francisella tularensis, Pasteurella multocida, Yersinia pestis, Bartonella bacilliformis, Bacteroides fragilis, Bartonella henselae, Streptobacillus moniliformis, Spirillum minus, Moraxella catarrhalis (Branhamella catarrhalis), and Shigella dysenteriae.

Other Gram-negative bacteria include spirochetes including, but not limited to, those belonging to the genera Treponema, Leptospira, and Borrelia. Particular spirochetes include, but are not limited to, Treponema palladium, Treponema pertenue, Treponema carateum, Leptospira interrogans, Borrelia burgdorferi, and Borrelia recurrentis.

Other Gram-negative bacteria include those of the order Rickettsiales including, but not limited to, those belonging to the genera Rickettsia, Ehrlichia, Orienta, Bartonella and Coxiella. Particular examples of such bacteria include, but are not limited to, Rickettsia rickettsii, Rickettsia akari, Rickettsia prowazekii, Rickettsia typhi, Rickettsia conorii, Rickettsia sibirica, Rickettsia australis, Rickettsia japonica, Ehrlichia chaffeensis, Orienta tsutsugamushi, Bartonella quintana, and Coxiella burni.

Gram-positive bacteria include those of the genera Listeria, Staphylococcus, Streptococcus, Bacillus, Corynebacterium, Peptostreptococcus, Actinomyces, Propionibacterium, Clostridium, Nocardia, and Streptomyces. Particular Gram-positive bacteria of interest include, but are not limited to, Listeria monocytogenes, Staphylococcus aureus, Streptococcus pyogenes, Streptococcus pneumoniae, Bacillus cereus, Bacillus anthracis, Clostridium botulinum, Clostridium perfringens, Clostridium difficile, Clostridium tetani, Corynebacterium diphtheriae, Corynebacterium ulcerans, Peptostreptococcus anaerobius, Actinomyces israeli, Actinomyces gerencseriae, Actinomyces viscosus, Actinomyces naeslundii, Propionibacterium propionicus, Nocardia asteroides, Nocardia brasiliensis, Nocardia otitidiscaviarum, and Streptomyces somaliensis.

Mycobacteria (e.g., those of the family Mycobacteriaceae) can also be used in the methods described herein. Particular mycobacteria include, but are not limited to, Mycobacterium tuberculosis, Mycobacterium leprae, Mycobacterium avium intracellulare, Mycobacterium kansasii, and Mycobacterium ulcerans.

Mycoplasma including, but not limited to, those of the genera Mycoplasma and Ureaplasma can be used in the methods described herein. Particular mycoplasma include, but are not limited to, Mycoplasma pneumoniae, Mycoplasma hominis, Mycoplasma genitalium, and Ureaplasma urealyticum.

A fungus can also be used in the methods described herein. Fungi include, but are not limited to, those belonging to the genera Aspergillus, Candida, Cryptococcus, Coccidioides, Sporothrix, Blastomyces, Histoplasma, Pneumocystis, and Saccharomyces. Particular fungi include, but are not limited to, Aspergillus fumigatus, Aspergillus flavus, Aspergillus niger, Aspergillus terreus, Aspergillus nidulans, Candida albicans, Coccidioides immitis, Cryptococcus neoformans, Sporothrix schenckii, Blastomyces dermatitidis, Histoplasma capsulatum, Histoplasma duboisii, and Saccharomyces cerevisiae.

A parasitic cell can also be used in the methods described herein. Parasitic cells include, but are not limited to, those belonging to the genera Entamoeba, Dientamoeba, Giardia, Balantidium, Trichomonas, Cryptosporidium, Isospora, Plasmodium, Leishmania, Trypanosoma, Babesia, Naegleria, Acanthamoeba, Balamuthia, Enterobius, Strongyloides, Ascaradia, Trichuris, Necator, Ancylostoma, Uncinaria, Onchocerca, Mesocestoides, Echinococcus, Taenia, Diphylobothrium, Hymenolepsis, Moniezia, Dicytocaulus, Dirofilaria, Wuchereria, Brugia, Toxocara, Rhabditida, Spirurida, Dicrocoelium, Clonorchis, Echinostoma, Fasciola, Fascioloides, Opisthorchis, Paragonimus, and Schistosoma. Particular parasitic cells include, but are not limited to, Entamoeba histolytica, Dientamoeba fragilis, Giardia lamblia, Balantidium coli, Trichomonas vaginalis, Cryptosporidium parvum, Isospora belli, Plasmodium malariae, Plasmodium ovale, Plasmodium falciparum, Plasmodium vivax, Leishmania braziliensis, Leishmania donovani, Leishmania tropica, Trypanosoma cruzi, Trypanosoma brucei, Babesia divergens, Babesia microti, Naegleria fowleri, Acanthamoeba culbertsoni, Acanthamoeba polyphaga, Acanthamoeba castellanii, Acanthamoeba astronyxis Acanthamoeba hatchetti, Acanthamoeba rhysodes, Balamuthia mandrillaris, Enterobius vermicularis, Strongyloides stercoralis, Strongyloides fulleborni, Ascaris lumbricoides, Trichuris trichiura, Necator americanus, Ancylostoma duodenale, Ancylostoma ceylanicum, Ancylostoma braziliense, Ancylostoma caninum, Uncinaria stenocephala, Onchocerca volvulus, Mesocestoides variabilis, Echinococcus granulosus, Taenia solium, Diphylobothrium latum, Hymenolepis nana, Hymenolepis diminuta, Moniezia expansa, Moniezia benedeni, Dicytocaulus viviparous, Dicytocaulus filarial, Dicytocaulus arnfieldi, Dirofilaria repens, Dirofilaria immitis, Wuchereria bancrofti, Brugia malayi, Toxocara canis, Toxocara cati, Dicrocoelium dendriticum, Clonorchis sinensis, Echinostoma, Echinostoma ilocanum, Echinostoma jassyenese, Echinostoma malayanum, Echinostoma caproni, Fasciola hepatica, Fasciola gigantica, Fascioloides magna, Opisthorchis viverrini, Opisthorchis felineus, Opisthorchis sinensis, Paragonimus westermani, Schistosoma japonicum, Schistosoma mansoni, Schistosoma haematobium, and Schistosoma haematobium.

A virus can also be used in the methods described herein. Viruses include, but are not limited to, those of the families Flaviviridae, Arenaviradae, Bunyaviridae, Filoviridae, Poxyiridae, Togaviridae, Paramyxoviridae, Herpesviridae, Picornaviridae, Caliciviridae, Reoviridae, Rhabdoviridae, Papovaviridae, Parvoviridae, Adenoviridae, Hepadnaviridae, Coronaviridae, Retroviridae, and Orthomyxoviridae. Particular viruses include, but are not limited to, Yellow fever virus, St. Louis encephalitis virus, Dengue virus, Hepatitis G virus, Hepatitis C virus, Bovine diarrhea virus, West Nile virus, Japanese B encephalitis virus, Murray Valley encephalitis virus, Central European tick-borne encephalitis virus, Far eastern tick-born encephalitis virus, Kyasanur forest virus, Louping ill virus, Powassan virus, Omsk hemorrhagic fever virus, Kumilinge virus, Absetarov anzalova hypr virus, Ilheus virus, Rocio encephalitis virus, Langat virus, Lymphocytic choriomeningitis virus, Junin virus, Bolivian hemorrhagic fever virus, Lassa fever virus, California encephalitis virus, Hantaan virus, Nairobi sheep disease virus, Bunyamwera virus, Sandfly fever virus, Rift valley fever virus, Crimean-Congo hemorrhagic fever virus, Marburg virus, Ebola virus, Variola virus, Monkeypox virus, Vaccinia virus, Cowpox virus, Orf virus, Pseudocowpox virus, Molluscum contagiosum virus, Yaba monkey tumor virus, Tanapox virus, Raccoonpox virus, Camelpox virus, Mousepox virus, Tanterapox virus, Volepox virus, Buffalopox virus, Rabbitpox virus, Uasin gishu disease virus, Sealpox virus, Bovine papular stomatitis virus, Camel contagious eethyma virus, Chamios contagious eethyma virus, Red squirrel parapox virus, Juncopox virus, Pigeonpox virus, Psittacinepox virus, Quailpox virus, Sparrowpox virus, Starlingpox virus, Peacockpox virus, Penguinpox virus, Mynahpox virus, Sheeppox virus, Goatpox virus, Lumpy skin disease virus, Myxoma virus, Hare fibroma virus, Fibroma virus, Squirrel fibroma virus, Malignant rabbit fibroma virus, Swinepox virus, Yaba-like disease virus, Albatrosspox virus, Cotia virus, Embu virus, Marmosetpox virus, Marsupialpox virus, Mule deer poxvirus virus, Volepox virus, Skunkpox virus, Rubella virus, Eastern equine encephalitis virus, Western equine encephalitis virus, Venezuelan equine encephalitis virus, Sindbis virus, Semliki forest virus, Chikungunya virus, O'nyong-nyong virus, Ross river virus, Parainfluenza virus, Mumps virus, Measles virus (rubeola virus), Respiratory syncytial virus, Herpes simplex virus type 1, Herpes simplex virus type 2, Varicella-zoster virus, Epstein-Barr virus, Cytomegalovirus, Human b-lymphotrophic virus, Human herpesvirus 7, Human herpesvirus 8, Poliovirus, Coxsackie A virus, Coxsackie B virus, ECHOvirus, Rhinovirus, Hepatitis A virus, Mengovirus, ME virus, Encephalomyocarditis (EMC) virus, MM virus, Columbia SK virus, Norwalk agent, Hepatitis E virus, Colorado tick fever virus, Rotavirus, Vesicular stomatitis virus, Rabies virus, Papilloma virus, BK virus, JC virus, B19 virus, Adeno-associated virus, Adenovirus, serotypes 3, 7, 14, 21, Adenovirus, serotypes 11, 21, Adenovirus, Hepatitis B virus, Coronavirus, Human T-cell lymphotrophic virus, Human immunodeficiency virus, Human foamy virus, Influenza viruses, types A, B, C, and Thogotovirus.

Examples of commensal organisms and symbionts include bacteria that make up the gut flora in mammals (e.g., humans).

### Samples for Analysis

The methods described herein can use any DNA sample containing target organism DNA, such as pathogen or parasite DNA, as well as contaminating DNA, for example, from a host organism. In particular embodiments, the samples used are biological samples (e.g., a fluid sample such as a blood sample or other cellular sample) taken from subjects (e.g., humans) that are infected with a particular parasite for analysis of the parasite genome.

The sample can contain any ratio by weight between the amount of parasite DNA and the amount of contaminating (e.g., host) DNA. For example the contaminating:parasite DNA ratio may be at least 500:1, 200:1, 150:1, 125:1, 100:1, 75:1, 60:1, 50:1, 40:1, 30:1, 25:1, 20:1, 15:1, 10:1, 5:1, 2:1, 1:1, 1:2, 1:5, 1:8, and 1:10.

The contaminating DNA may be from any source. In certain situations, the contaminating DNA is from the host organism infected with the parasite or pathogen, or a DNA from a symbiotic or commensal species.

The methods disclosed herein have been validated using two approaches. First, mock clinical samples containing both parasite (P. falciparum) DNA were mixed with Homo sapiens DNA at a ratio of 99:1 (H sapiens:P. falciparum) to generate samples. Samples were fluorescently quantitated prior to mixing using a PicoGreen assay (Singer et al., Anal. Biochem. 1997, 249:228-238). Authentic clinical samples were collected in 2008 from symptomatic patients at a clinic in Thies, Senegal under an approved IRB protocol. Samples consisted of whole blood dried and stored on a Whatman FTA card and/or frozen whole blood stored in a glycerolyte 57 solution. DNA was extracted using a DNeasy kit (Qiagen).

### Baits

The methods disclosed herein employ nucleic acid baits that provide significant coverage of the parasite (or pathogen, commensal organism, or symbiont) genome. The baits must be of sufficient length to provide specificity to the organism's genome. As explained below, baits of either 140 bases or about 250 bases have been used successfully; however, any length (e.g., at least 50, 60, 70, 80, 90, 100, 110, 120, 130, 140, 150, 175, 200, 225, 250, 300, or 350 bases) that provides sufficient specificity can be used in the methods of the present invention. The baits, in certain embodiments, may be DNA or RNA.

Bait sequences can be generated from any appropriate source, for example from genomic information, from cDNA sequences, or from the whole genome of the organism being targeted. As explained below, the methods can employ synthetic oligonucleotides or sheared genomic DNA.

Synthetic oligonucleotides are generated, for example, where the genome of the target organism has already been sequenced. In this situation, a number of oligonucleotides that provide the desired genome coverage can be designed. Such sequences typically will lack homology to the contaminating (e.g., host) DNA. Any appropriate number of oligonucleotides can be used. In the example described below, nearly 25,000 oligonucleotides were used; however, the skilled artisan will be able to determine an appropriate number. In certain cases, fewer oligonucleotides may be used (e.g., about or at least 22,000, 20,000, 18,000, 15,000, 12,000, 10,000, 8000, 6000, 5000, 4000, 3000, 2000, 1000, or 500 oligonucleotides). In other cases, larger number of oligonucleotides may be desirable (e.g., about or at least 28,000, 30,000, 35,000, 40,000, 45,000, 50,000 or 60,000 oligonucleotides. The bait sequences, if desired, can be labeled using PCR (e.g., with detectably labeled primers, such as biotinylated primers) or can be converted into labeled (e.g., biotinylated) RNA sequences using art-recognized methods such as incorporation of biotinylated nucleotides.

In one example using synthetic oligonucleotides as bait, synthetic 140 bp oligonucleotides were obtained from Agilent and designed to capture exonic regions of the P. falciparum genome as defined in the 3D7 v.5.0 reference assembly. The final bait set included 24,246 oligonucleotides (3.4 Mb) with unique BLAT matches to the P. falciparum 3D7 reference genome assembly and no homology to the human genome. To generate synthetic single-stranded biotinylated RNA bait in vitro transcription was performed with biotin-labeled UTP using the MEGAshortscript T7 kit (Ambion) as described previously (Gnirke et al., Nat. Biotechnol. 2009, 27:182-189).

Another approach is to use the pathogen genome itself as WGB to generate the baits used in the methods described herein. Here, genomic DNA from the pathogen is processed into smaller pieces using any technique known in the art, such as shearing. Shearing can be controlled to ensure that particular size fragments are generated. In one example, fragments of about 250 bp in length were produced, although the skilled artisan would readily be able to determine appropriate lengths for such fragments. Following fragmentation, various steps, including end repair, addition of adapters, and clean up (e.g., using Qiagen kits) can then be performed. Amplification of the DNA can be performed by PCR. RNA promoters (e.g., the T7 promoter) or other functional sequences can also be added, e.g., as part of the adapter sequence or by further PCR. Labeled RNA can be generated, for example, by transcribing the RNA in the presence of labeled nucleotides. Additional approaches for bait sequence design are described in PCT Publication WO 2009/099602.

In one example, WGB was generated by shearing 3 μg of P. falciparum 3D7 DNA for 4 min using a Covaris E210 instrument set to duty cycle 5, intensity 5, and 200 cycles per burst. The mode of the resulting fragment-size distribution was 250 bp. End repair, addition of a 3′-A, adaptor ligation, and reaction clean-up followed the Illumina's genomic DNA sample preparation kit protocol except that adapter consisted of oligonucleotides 5′-TGTAACATCACAGCATCACCGCCATCAGTCxT-3′ (“x” refers to an exonuclease I-resistant phosphorothioate linkage) (SEQ ID NO:1)and 5′-[PHOS]GACTGATGGCGCACTACGACACTACAATGT-3′ (SEQ ID NO:2). The ligation products were purified (Qiagen), amplified by 8-12 cycles of PCR on an ABI GeneAmp 9700 thermocycler in Phusion High-Fidelity PCR master mix with HF buffer (NEB) using PCR forward primer 5′-CGCTCAGCGGCCGCAGCATCACCGCCATCAGT-3′ (SEQ ID NO:3) and reverse primer 5′-CGCTCAGCGGCCGCGTCGTAGTGCGCCATCAGT-3′ (SEQ ID NO:4). Initial denaturation was 30 s at 98° C. Each cycle was 10 s at 98° C., 30 s at 50° C. and 30 s at 68° C. PCR products were size-selected on a 4% NuSieve 3:1 agarose gel followed by QlAquick gel extraction. To add a T7 promoter, size-selected PCR products were re-amplified as above using the forward primer 5′-GGATTCTAATACGACTCACTATACGCTCAGCGGCCGCAGCATCACCGCCATCAGT -3′ (SEQ ID NO:5). Qiagen-purified PCR product was used as template for Whole Genome biotinylated RNA Bait preparation with the MEGAshortscript T7 kit (Ambion) (Gnirke et al., Nat. Biotechnol. 2009, 27:182-189).

### Whole Genome Amplification

Prior to hybridization, it may be desirable to increase the amount of DNA in the sample for analysis. Any technique for WGA may be used. The hybrid selection protocol requires a minimum of 2 μg of input DNA (combined host and pathogen), a quantity which may not be available from many types of field samples. Therefore, we also performed hybrid selection with both bait classes on 2 μg of whole-genome-amplified DNA generated from 10 ng of the mock clinical sample. qPCR analysis indicated that WGA does not significantly alter the fraction of malaria DNA present in the sample (post WGA % P. falciparum DNA=1.1+/−0.1).

WGA can be performed using any technique known in the art. See, e.g., Hosono et al. Genome Res. 2003, 13:954-64; Wells et al., Nucl. Acids Res. 1999, 27: 1214-18; Cheung et al., Proc. Natl. Acad. Sci. USA 1996, 93:14676-9; and Lasken et al., Trends Biotechnol. 2003, 21:531-5. Kits for performing WGA are available commercially, e.g., from Qiagen (REPLI-g UltraFast Mini Kit; catalog Nos. 150033 and 150035; REPLI-g Mini and Midi Kits, catalog Nos. 150090, 150043, 150045, 150023, and 150025) Sigma-Aldrich (GenomePlex® Whole Genome Amplification Kit, catalog No. WGA1; GenomePlex® Complete Whole Genome Amplification Kit, catalog No. WGA2), and Active Motif (GenoMatrix™ Whole Genome Amplification Kit; catalog No. 58001). The experiments described herein were performed WGA using the Repli-G kit available from Qiagen.

### Sample Preparation

Prior to hybridization, the sample containing the DNA sample may be prepared by end labeling for sequencing and/or other analytical purposes, using the general approach described in Gnirke et al., Nat. Biotechnol. 2009, 27:182-189. In one example, whole-genome fragment libraries were prepared using a modification of Illumina's genomic DNA sample preparation kit. Briefly, 3 μg of the sample DNA was sheared for 4 min. on a Covaris E210 instrument set to duty cycle 5, intensity 5, and 200 cycles per burst. The mode of the resulting fragment-size distribution was ˜250 bp. End repair, non-templated addition of a 3′-A, adapter ligation, and reaction clean-up followed the kit protocol except that we used a generic adapter for libraries destined for shotgun sequencing after hybrid selection. This adapter consisted of oligonucleotides C (5′-TGTAACATCACAGCATCACCGCCATCAGTCxT-3′ with “x” denoting a phosphorothioate bond resistant to excision by 3′-5′ exonucleases) (SEQ ID NO:1) and D (5′-[PHOS] GACTGATGGCGCACTACGACACTACAATGT-3′) (SEQ ID NO:2). The ligation products were purified(Qiagen) and size-selected on a 4% NuSieve 3:1 agarose gel followed by QIAquick gel extraction. A standard preparation starting with 3 μg of genomic DNA yielded ˜500 ng of size selected material with genomic inserts ranging from ˜200 to ˜350 bp, i.e., enough for one hybrid selection. To increase yield, an aliquot was amplified by 12 cycles of PCR in Phusion High-Fidelity PCR master mix with HF buffer (NEB) using Illumina PCR primers 1.1 and 2.1, or, for libraries with generic adapters, oligonucleotides C and E (5′-ACATTGTAGTGTCGTAGTGCGCCATCAGTCxT-3′) (SEQ ID NO:6) as primers. After QlAquick cleanup, if necessary, fragment libraries were concentrated in a vacuum microfuge to 250 ng per μl before hybrid selection.

### Hybridization

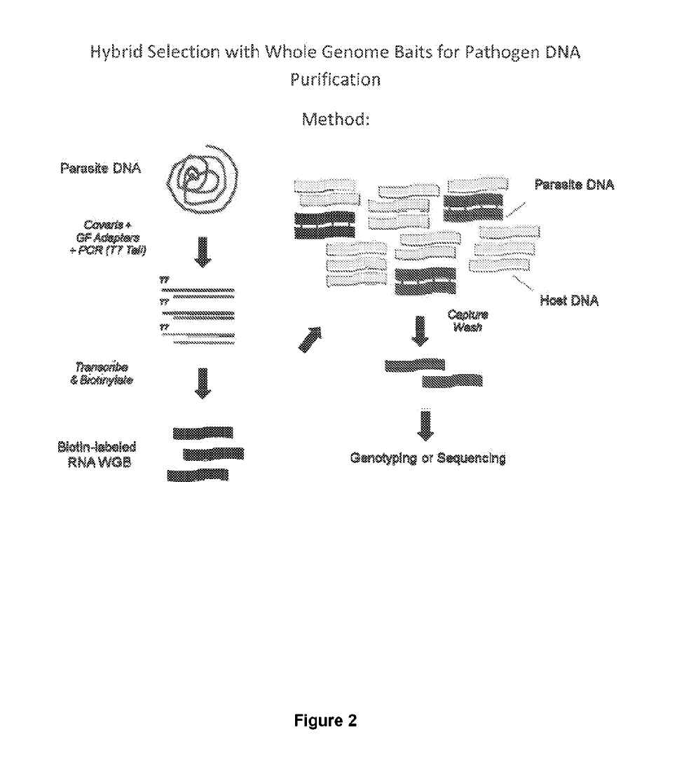

Hybridization between the test sample and the bait sequence is conducted under any conditions in which the bait sequences hybridize to the target organism's DNA (e.g., pathogen, commensal organism, or symbiont DNAs), but do not substantially hybridize to the contaminating DNA. This can involve selection under high stringency conditions. Following hybridization, the labeled baits can be separated based on the presence of the detectable label, and the unbound sequences are removed under appropriate wash conditions that remove the nonspecifically bound DNA, but do not substantially remove the DNA that hybridizes specifically. Exemplary hybridization schemes are shown in FIGS. 1 and 2.

In one example, hybrid selection using either synthetic bait or WGB was carried out as described previously (Gnirke et al., Nat. Biotechnol. 2009, 27:182-189 and PCT Publication WO 2009/099602) and detailed below.

Hybridization was conducted at 65° C. for 66 h with 500 ng of “pond” (i.e., target) libraries carrying standard or indexed Illumina paired-end adapter sequences, as explained above, and 500 ng of bait in a volume of 30 After hybridization, captured DNA was pulled down using streptavidin Dynabeads (Invitrogen). Beads were washed once at room temperature for 15 min with 0.5 ml 1×SSC/0.1% SDS, followed by three 10-min. washes at 65° C. with 0.5 ml pre-warmed 0.1×SSC/0.1% SDS, re-suspending the beads once at each washing step. Hybrid-selected DNA was eluted with 50 μl 0.1 M NaOH. After 10 min. at room temperature, the beads were pulled down, the supernatant transferred to a tube containing 70 μl of 1 M Tris-HCl, pH 7.5, and the neutralized DNA desalted and concentrated on a QIAquick MinElute column and eluted in 20 μl.

This protocol was optimized by exploring two different hybridization temperatures (60° C. vs. 65° C.) and four different wash stringencies (0.1×SSC, 0.25×SSC, 0.5×SSC, and 0.75×SSC). Eight mock clinical samples were hybridized with WGB and washed under all combinations of the above conditions. Enrichment was measured by qPCR and sequencing (one indexed Illumina GAIIx lane). The best enrichment was observed under the standard high stringency conditions used for all previously reported experiments (hybridization at 65° C. and high stringency wash (0.1×SSC). Results are presented in Table 2.

TABLE 2
qPCR enrichment measurements

| | | | Pre | Post | |
|---|---|---|---|---|---|
| | | | Hyb Sel | Hyb Sel | |
| Hyb | | | [DNA] | [DNA] | Fold |
| Temperature | Stringency | Wash | (pg/μl) | (pg/μl) | Enrichment |
| 65° C. | High | 0.10 × SSC | 10.0 | 342.9 | 34.3 |
| | Med/High | 0.25 × SSC | 10.0 | 258.2 | 25.8 |
| | Med/Low | 0.50 × SSC | 10.0 | 227.9 | 22.8 |
| | Low | 0.75 × SSC | 10.0 | 181.4 | 18.1 |
| 60° C. | High | 0.10 × SSC | 10.0 | 288.6 | 28.9 |
| | Med/High | 0.25 × SSC | 10.0 | 232.9 | 23.3 |
| | Med/Low | 0.50 × SSC | 10.0 | 203.5 | 20.4 |
| | Low | 0.75 × SSC | 10.0 | 196.3 | 19.6 |

### Analysis of Enrichment

To confirm that the hybridization results in enrichment of the target organism DNA, any method known in the art, including quantitative PCR (qPCR), can be used.

Sequencing of the hybrid selected samples revealed a significant increase in representation of Plasmodium DNA in every case. The synthetic baits respectively yielded an average of 41-fold and 44-fold parasite DNA enrichment for unamplified and WGA simulated clinical samples in genomic regions targeted by the baits, as measured by qPCR. WGB yielded parasite genome-wide average enrichment levels of 37-fold and 40-fold for the unamplified and WGA input samples, respectively.

Enrichment of malaria DNA in samples was assessed using a panel of malaria qPCR primers designed to conserved regions of the P. falciparum 3D7 v.5.0 reference genome. Enrichment for each amplicon was calculated as the ratio between the amount of DNA presented pre and post hybrid selection, with cT counts corrected for qPCR efficiency using a standard curve for each amplicon. All qPCR reactions utilized 1 μl of template containing 1 ng of total DNA. Estimated enrichment for the samples was calculated as the mean enrichment observed across all tested amplicons. Quantitation of human DNA in the clinical samples was performed prior to sequencing using the Taqman RNase P Detection Reagents kit (Applied Biosystems).

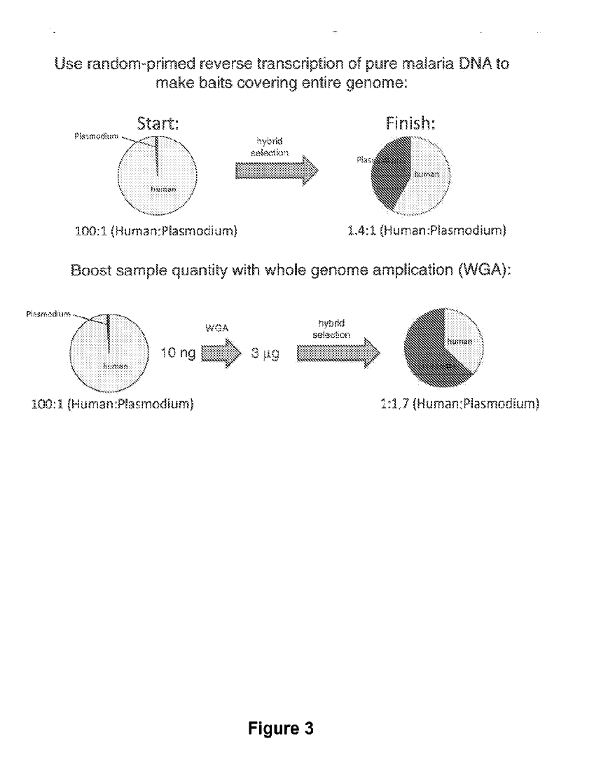

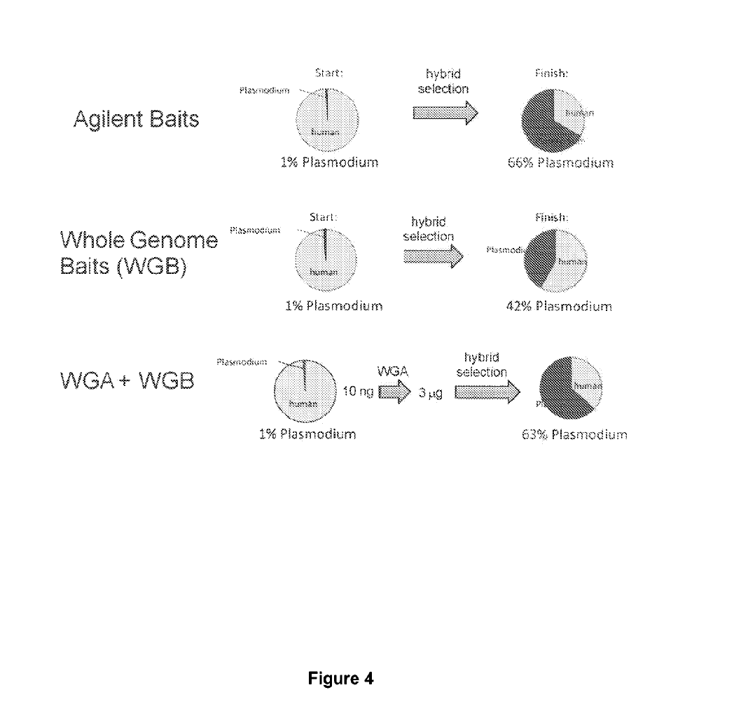

Exemplary results from hybridization are shown in FIGS. 3 and 4.

### Sequencing

Following hybridization, the captured target organism DNA can be sequenced by any means known in the art. Sequencing of nucleic acids isolated by the methods described herein is, in certain embodiments, carried out using massively parallel short-read sequencing (e.g., the Solexa sequencer, Illumina Inc., San Diego, Calif.), because the read out generates more bases of sequence per sequencing unit than other sequencing methods that generate fewer but longer reads. However, sequencing also can be carried out using other methods or machines, such as the sequencers provided by 454 Life Sciences (Branford, Conn.), Applied Biosystems (Foster City, Calif.; SOLiD sequencer), or Helicos BioSciences Corporation (Cambridge, Mass.), or by standard Sanger dideoxy terminator sequencing methods and devices.

Each sample was sequenced using one lane of Illumina 76 bp paired-end reads. The libraries of pure P. falciparum DNA and hybrid selected artificial clinical samples were each sequenced with one Illumina GAIIx lane. The hybrid selected authentic clinical sample (Th231.08) was sequenced with one Illumina HiSeq lane. Sequence data have been deposited in the NCBI Short Read Archive under Project IDs 51255 & 43541.

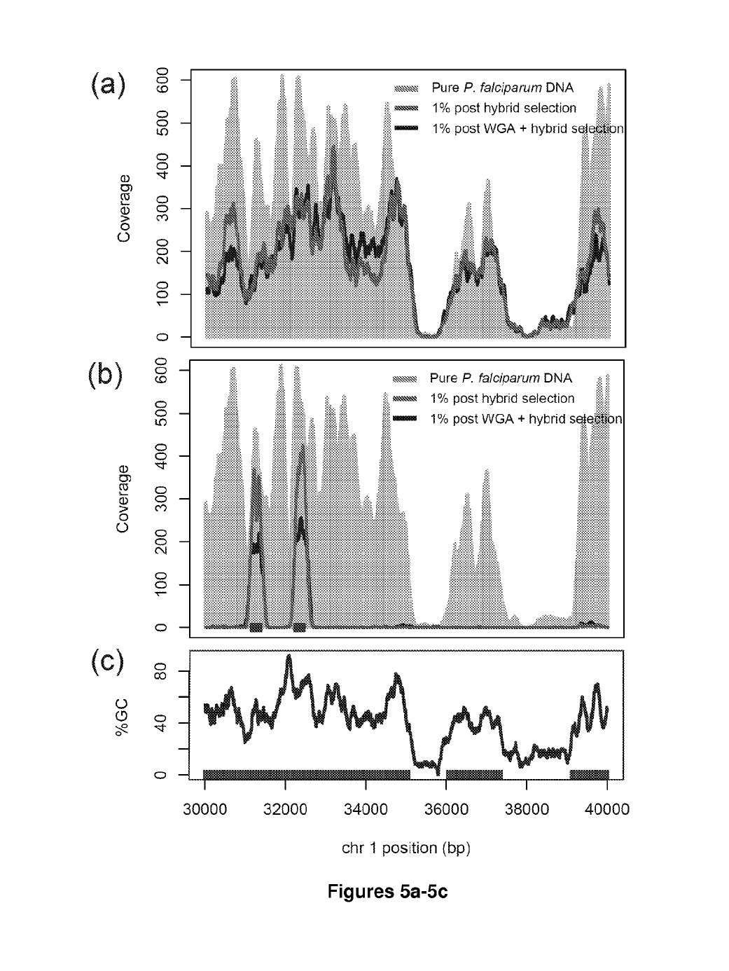

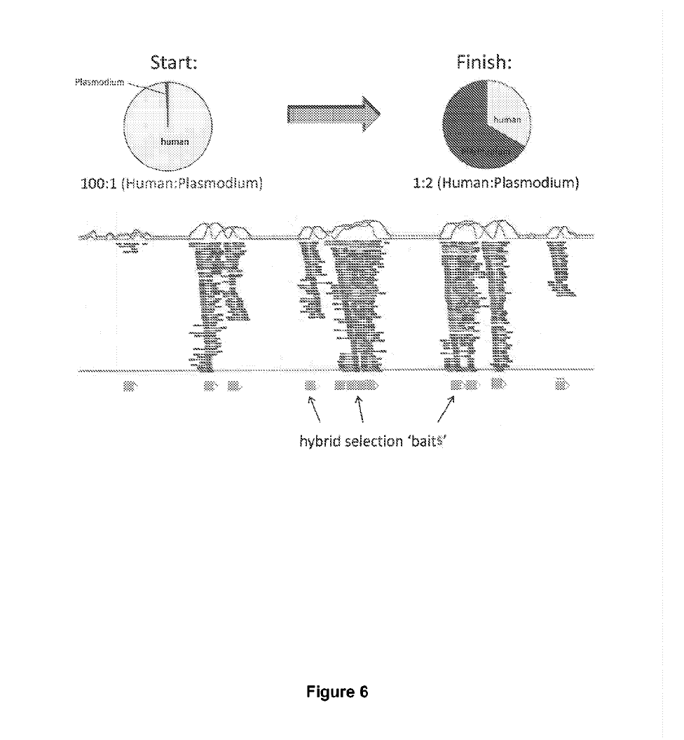

Illumina sequencing coverage in the WGB hybrid selected samples is correlated with GC content, mirroring what is observed in sequencing data from pure P. falciparum DNA (FIG. 5a). With a genome-wide A/T composition of 81% (Gardner et al., Nature 2002, 419:498-511), achieving uniform sequencing coverage of the P. falciparum genome is challenging even under ideal circumstances. No reduction in coverage uniformity as a result of the hybrid selection process was observed. WGA did not compromise mean genome-wide sequencing coverage relative to unamplified input DNA (67.5× vs. 67.1× for a single Illumina GAIIx lane, respectively). Sequencing coverage of the samples hybrid selected using synthetic 140 bp baits was tightly localized to the genomic regions to which baits were designed (FIG. 5b). Coverage levels in baited regions that were significantly higher than what is observed from comparable sequencing of pure P. falciparum DNA. This indicates that hybrid selection with synthetic baits may be useful not only for reducing off-target coverage in the host genome, but also for strategically augmenting coverage levels in regions of pathogen genomes where heightened sequence coverage could be informative, such as highly polymorphic antigenic regions subject to host immune pressure. Results of such sequencing are shown in FIG. 6.

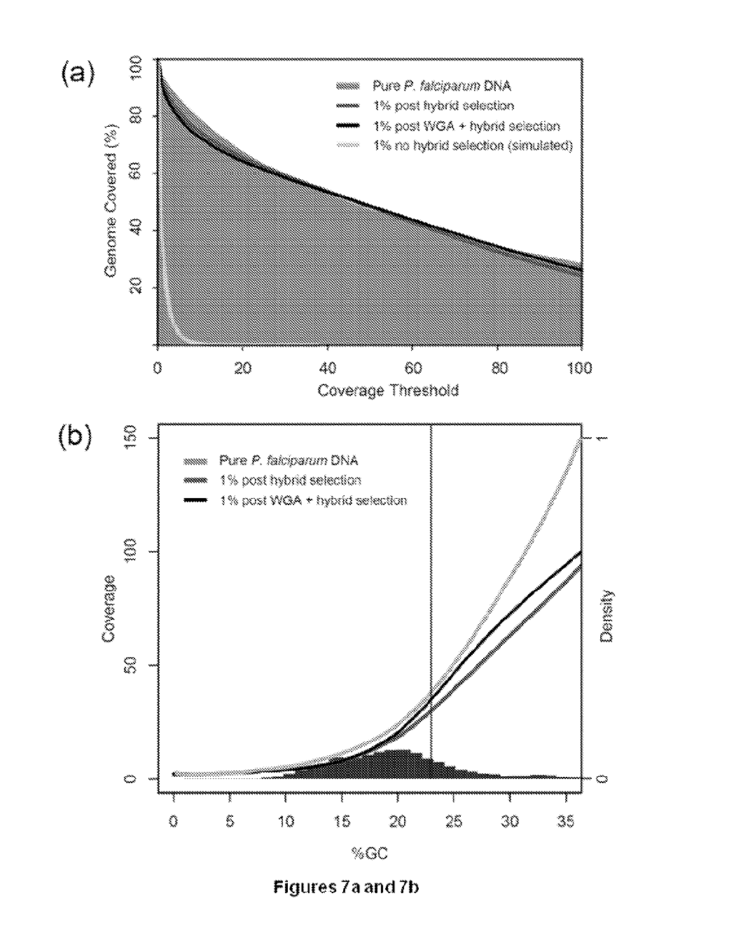

Though effective sequencing coverage levels are reduced in the hybrid-selected mock clinical samples relative to pure P. falciparum DNA due to the incomplete elimination of human DNA, this reduction is small compared to the 100-fold reduction in coverage expected without hybrid selection. Genome-wide coverage is depicted in FIG. 7a, which illustrates that the extent of the genome covered to various thresholds is highly similar for the pure P. falciparum and hybrid selected mock clinical samples, and significantly higher than simulated coverage levels we would have predicted to be observed from sequencing an unpurified version of the sample. Genome-wide coverage levels as a function of local % GC (% G+C) are plotted in FIG. 7b for the WGB experiments. The relationship between % GC and coverage observed in whole genome shotgun sequencing data is decreased by hybrid selection due to reduced coverage in rare high % GC genomic regions (Spearman's rₛ for % GC vs. coverage of pure malaria DNA: 0.86; vs. WGB hybrid selected DNA: 0.59; vs. WGA+WGB hybrid selected DNA: 0.64). The vertical line in FIG. 7b represents the average % GC of exonic sequence (23%). Assuming a minimum threshold of 10-fold sequencing coverage is required for accurate SNP calling, 99.2% of exonic bases exhibited this coverage or greater in reads generated from the pure P. falciparum DNA sample. The unamplified and amplified hybrid selected samples achieved at least 10-fold coverage for 98.3% and 98.0% of exonic bases, respectively. This indicates that sequencing data generated from hybrid selected clinical samples is likely as useful as data generated from pure pathogen DNA samples for downstream analyses.

### Data Analysis

Quality scores on Illumina reads were resealed using the MAQ sol2sanger utility (Li et al., Genome Res. 2008, 18:1851-1858). Reads were then aligned to P. falciparum 3D7 (PlasmoDB 5.0) using BWA (Li et al., Bioinformatics 2009, 25:1754-1760). Sequenced reads were sorted and the consensus sequence was determined using the SAMtools utilities (Li et al., Bioinformatics 2009, 25:2078-2079). % GC was calculated from 140 by windows across the P. falciparum genome.

The human:P. falciparum DNA ratio in each sequence dataset was estimated from sequencing data by randomly sampling 50 K pairs of mated reads and measuring the fractions that uniquely mapped to human vs. P. falciparum reference genome assemblies.

Principal components analysis was performed using Eigensoft software (Patterson et al., PLoS Genet. 2006, 2:e190) on 8,300 non-singleton SNPs with coverage of at least 10-fold in all strains and consensus quality scores of at least 30.

### Compositions, Kits, and Systems

As described herein, the invention features compositions, kits, and systems related to the methods described herein. The compositions include WGB. The kits include WGB, or reagents suitable for producing WGB, along with other reagents, such as a solid phase containing a binding partner of the detectable label on the WGB or an RNA polymerase. The kits may also include solutions for hybridization, washing, or eluting of the DNA/solid phase compositions described herein, or may include a concentrate of such solutions.

The invention also features systems capable of carrying out the methods described herein.

The follow example is intended to illustrate, rather than limit, the invention.

### EXAMPLE 1

### Hybrid Selection on Authentic Clinical Samples

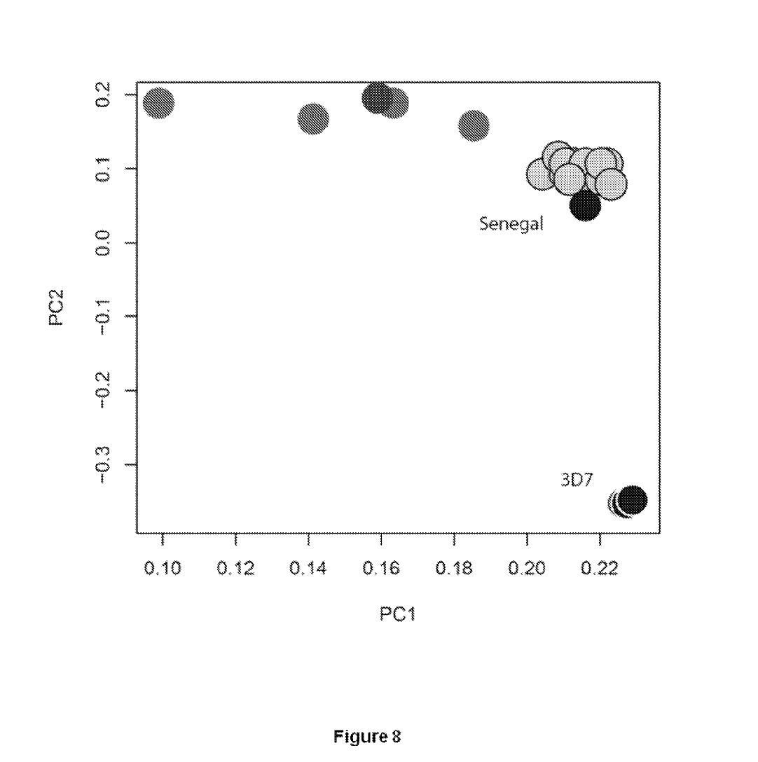

To test this application, we performed WGA and hybrid selection on DNA extracted from a clinical P. falciparum sample (Th231.08) collected on filter paper in Thies, Senegal in 2008 and stored at room temperature for over a year. By qPCR, the Plasmodium DNA in the original sample was estimated to comprise approximately 0.11% of the total DNA by mass. Following WGA and hybrid selection, Plasmodium DNA represented 7.7% of total DNA present, an approximately 70-fold increase in parasite DNA representation. Illumina HiSeq sequencing data confirmed that at least 5.9% of map-able reads in the hybrid selected sample corresponded to Plasmodium. The fraction of human reads after hybrid selection remained high due to the extreme initial ratio of host:parasite DNA, but the enrichment factor in this case was sufficient to rescue the feasibility of sequencing this sample. A total of 26,366 single nucleotide polymorphisms (SNPs) were identified relative to the P. falciparum reference assembly (more than 1 per kb), close to the number of SNPs identified (33,094 - 41,123) from 11 other culture-adapted Senegalese parasite lines sequenced without hybrid selection. Principal components analysis of SNP genotypes confirms the similar genomic profile of the hybrid selected and non-hybrid selected Senegalese strains, as well as hybrid selected and non-hybrid selected 3D7 reference strain datasets generated from sequencing the mock clinical samples (FIG. 8). Despite the use of WGB generated from the 3D7 reference genome, the DNA captured from the Senegal isolate has the SNP profile of Senegal DNA, rather than 3D7 DNA, suggesting that polymorphisms do not strongly bias enrichment. In addition, the highly polymorphic regions of the isolate did not suffer a relative drop in sequencing coverage after hybrid selection. Hybrid selection of a panel of 12 other clinical malaria samples from Senegal yielded an average of 35-fold enrichment, as measured by qPCR (Table 1), with enrichment amount inversely proportional to the initial fraction of parasite DNA in the samples.

A second round of hybrid selection was conducted on the Th231.08 clinical sample to determine whether Plasmodium DNA titer could be boosted above approximately 7%. The second round of hybrid selection was carried out under identical hybridization and wash conditions. qPCR analysis indicates this yielded a sample in which 47.5% of the genetic material was Plasmodium by mass (a 6.7 fold enrichment). This lower fold enrichment is consistent with our previous observation that fold enrichment is inversely proportional to initial parasite DNA titer, but in this case yields a sample highly amenable to cost-efficient and deep sequencing.

### Other Embodiments

All patents, patent applications, and publications mentioned in this specification are herein incorporated by reference to the same extent as if each independent patent, patent application, or publication was specifically and individually indicated to be incorporated by reference.

What is claimed is:

1. A method for enriching the genome of a target organism in a DNA sample that includes both contaminating DNA and DNA of said target organism, said method comprising:
(a) contacting said sample with at least 1,000 different, detectably-labeled hybridization bait sequences specific for said target organism DNA, said bait sequences being prepared from the whole genome of the target organism, under conditions in which said bait sequences hybridize to said target organism DNA but do not substantially hybridize to said contaminating DNA; and
(b) selectively isolating said hybridized target organism DNA based on said detectable label, thereby enriching for said genome of said target organism.

2. A method of genotyping or sequencing the genome of a target organism, said method comprising sequencing at least a portion of the genome in a sample containing DNA from a target organism prepared according to claims 1.

3-4. (canceled)

5. The method of claim 1 any of claims 1 1, wherein said DNA sample, prior to step (a) contacting, is subject to shearing and end-labeling.

6. (canceled)

7. The method of claim 1, wherein most of the DNA in said DNA sample is contaminating DNA or the ratio of contaminating DNA to target DNA is at least 10:1, at least 40:1, or at least 80:1.

8-10. (canceled)

11. The method of claim 1, wherein said bait sequences are prepared by a method that comprises fragmenting genomic DNA of said target organism, and optionally are end-labeled with oligonucleotide sequences suitable for PCR amplification or DNA sequencing.

12. (canceled)

13. The method of claim 11, wherein said bait sequences are prepared by a method including attaching an RNA promoter sequence to said genomic DNA fragments and preparing said bait by transcribing said DNA fragments into RNA, wherein optionally said transcription includes the use of biotinylated nucleotides and/or biotinylated primers and/or said bait sequences are generated by nick-translation labeling of purified target organism DNA with biotinylated deoxynucleotides.

14. (canceled)

15. The method of claim 1, wherein said bait sequences are prepared from specific regions of the target organism genome.

16. The method of claim 15, wherein said bait sequences are prepared synthetically.

17. The method of claim 1, wherein said bait sequences are labeled with biotin, a hapten, or an affinity tag.

18-19. (canceled)

20. The method of claim 1, wherein said target organism DNA is captured using a streptavidin molecule attached to a solid phase.

21-23. (canceled)

24. The method of claim 1, wherein said sample is contacted with at least 5,000, 10,000, or 20,000 different bait sequences.

25-26. (canceled)

27. The method of claim 1, wherein said bait sequences are 60-500 bp or 100-300 bp in length.

28. (canceled)

29. The method of claim 1, wherein prior to performing step (a), whole genome amplification is performed on said DNA sample.

30-31. (canceled)

32. The method of claim 1, wherein said target organism is a eukaryote (optionally a fungus or a parasite), a prokaryote (optionally a bacterium), an archeal organism, or a virus.

33-37. (canceled)

38. The method of claim 32, wherein said target organism is Plasmodium vivax, Plasmodium falciparum, Plasmodium ovale, Plasmodium malariae, Chlamydia trachomatis, Trypanosoma cruzi, or Wolbachia.

39. The method of claim 1, wherein said contaminating DNA is host DNA, which is optionally mammalian DNA, such as human DNA.

40-41. (canceled)

42. The method of claim 1, wherein said DNA sample is a biological sample, which optionally is a cell sample, a blood sample, or contains blood components, and wherein optionally the sample is taken from a human infected with, or suspected of being infected with, a parasite or a pathogen.

43-44. (canceled)

45. A method for preparing whole genome bait, said method comprising:
(a) transcribing RNA from fragmented genomic DNA of an organism, said DNA containing adapter sequences that comprise an RNA polymerase start site and optionally being sheared (e.g., to an average of 100-500 or 250 bases in length); and
(b) detectably labeling said RNA, thereby preparing whole genome bait.

46-55. (canceled)

56. A composition comprising whole genome baits produced by the method of claim 1.

57. A composition comprising RNA molecules that:
(a) are detectably labeled;
(b) are 100-1000 bases in length;
(c) together cover at least 50%, 95%, or 99% of the genome of a target organism.

58-59. (canceled)

60. A kit comprising:
(a) a composition of claim 57; and
(b) a solid phase, wherein a binding partner of said detectable label is attached to said solid phase.

61-69. (canceled)


SEQUENCE LISTING (6 sequences)

SEQ ID NO: 1 - DNA, 31 bp, Artificial Sequence
  Synthetic Construct
tgtaacatcacagcatcaccgccatcagtct

SEQ ID NO: 2 - DNA, 30 bp, Artificial Sequence
  Synthetic Construct
gactgatggcgcactacgacactacaatgt

SEQ ID NO: 3 - DNA, 32 bp, Artificial Sequence
  Synthetic Construct
cgctcagcggccgcagcatcaccgccatcagt

SEQ ID NO: 4 - DNA, 33 bp, Artificial Sequence
  Synthetic Construct
cgctcagcggccgcgtcgtagtgcgccatcagt

SEQ ID NO: 5 - DNA, 55 bp, Artificial Sequence
  Synthetic Construct
ggattctaatacgactcactatacgctcagcggccgcagcatcaccgccatcagt

SEQ ID NO: 6 - DNA, 31 bp, Artificial Sequence
  Synthetic Construct
acattgtagtgtcgtagtgcgccatcagtct